(19) United States
(12) Patent Application Publication
(10) Pub. No.: US 2016/0329588 A1
(43) Pub. Date: Nov. 10, 2016

(54) A METHOD FOR THE RECOVERY OF ORGANIC COMPOUNDS FROM WASTEWATER FOR GENERATING ELECTRICITY

(71) Applicant: UNITED UTILITIES PLC, Great Sankey, Warrington (GB)

(72) Inventor: MINH SON LE, Great Sankey, Warrington (GB)

(73) Assignee: UNITED UTILITIES PLC, Great Sankey, Warrington (GB)

(21) Appl. No.: 15/109,542

(22) PCT Filed: Jan. 9, 2015
(86) PCT No.: PCT/GB2015/050036

§ 371 Date: Jul. 1, 2016

(30) Foreign Application Priority Data
Jan. 9, 2014 (GB) 1400349.5

(51) Int. Cl. H01M 8/16; C02F 1/24; C02F 1/38; C12P 7/64; C02F 3/12; C02F 3/28; C02F 11/04; C02F 9/00; C02F 1/44

(52) U.S. Cl. CPC H01M 8/16; C02F 9/00; C02F 1/24; C02F 1/385; C02F 1/444; C02F 3/12; C02F 3/28; C02F 11/04; C12P 7/6409; C02F 2303/06; C02F 2305/06; C02F 2101/30

(57) ABSTRACT

The present invention relates to a method of recovering organic compounds from wastewater for use in electricity generation in a microbial fuel cell, comprising the steps of: converting organic compounds from wastewater into a biomass; recovering the biomass from the wastewater; followed by substantially dissolving or breaking down the biomass to form a cell lysate; fermenting the cell lysate to form volatile fatty acids (VFA) in a broth; and separating the VFA in the VFA broth to produce a clarified VFA stream.

The present invention relates to a method for the recovery of organic compounds from wastewater in high concentration. More specifically the present invention relates to a method for the recovery of organic compounds such as volatile fatty acids (VFA) in high concentration from wastewater for use in a microbial fuel cell (MFC). In addition, the present invention relates to a method for the generation of electricity using organic compounds such as volatile fatty acids (VFA) from wastewater using a microbial fuel cell (MFC).

When micro-organisms consume a substrate such as sugar under aerobic conditions carbon dioxide and water are produced. In contrast, in the absence of oxygen, micro-organisms produce carbon dioxide, protons and electrons as described in equation 1 below:

C₁₂H₂₂O₁₁+13H_{2O→12}CO₂+48H⁺+48e⁻ Equation 1.

A microbial fuel cell (MFC) is a device that converts chemical energy into electrical energy using microorganisms as described below. That is, in a microbial fuel cell (MFC), specific types of microorganisms, typically bacteria, break down organic material, such as that found in wastewater, at an anode under anaerobic (without oxygen) conditions.

The organic material is broken down in solution by the bacteria, and the bacteria release electrons (negatively charged particles), protons (positively charged hydrogen ions) and carbon dioxide into the solution. The anode collects the electrons, which then travel to a cathode via an external circuit (that is an electric current flows between the cathode and anode). The protons travel through solution in the microbial fuel cell to the cathode. The carbon dioxide may be captured and reused. Consequently, in an MFC, electricity is produced and extracted from the electron-carrying external circuit. The electrons arriving at the cathode under aerobic conditions, (that is, in the presence of oxygen) combine with the protons and oxygen, typically from air, to form water.

A typical MFC system therefore comprises anode and cathode compartments separated by a cation specific membrane. In the anode compartment, fuel is oxidized by the microorganisms, generating electrons and protons. Electrons are transferred to the cathode compartment through an external electric circuit, and the protons are transferred to the cathode compartment through the membrane. Electrons and protons are consumed in the cathode compartment, combining with oxygen to form water.

In Australia, Foster's Brewing Company has successfully demonstrated the use of a MFC to convert brewery wastewater into carbon dioxide, clean water, and electricity. However, the practical commercial application of MFC technology requires both efficient and cost effective electrodes as well as a suitable feed stock. The performance and cost of electrodes are therefore most important aspects in the design of MFC reactors. Consequently, a wide range of electrode materials and configurations have been tested and developed in recent years to improve MFC performance and lower material cost.

In addition, anodic electrode surface modifications have been widely used to improve bacterial adhesion and electron transfer from bacteria to the electrode surface.

In contrast, the effect, type and concentration of suitable substrates on power generation has received much less attention. Indeed, to date, most researchers in their investigations of MFC applications have concentrated on relatively simple organic substrates which are mostly water soluble substrates.

The substrates used in MFC to date range from: carbohydrates (glucose, sucrose, cellulose, and starch); volatile fatty acids (VFA) (formate, acetate, and butyrate); alcohols (ethanol, methanol); to amino acids; proteins; and even inorganic components such as sulfides or acidic mine drainages.

Although the use of a pure or single substrate allows the study of metabolic processes and conversion products during the microbial conversion, it is not feasible to power full scale microbial fuel cells with pure substrates from an economical point of view. Consequently, the use of second generation bio-fuels or organic waste streams provides a highly promising choice for microbial fuel cells because the use of same provides an actual treatment of problem waste streams with the benefits of energy generation.

A number of investigators have reported that more complex organic material containing a large variety of different readily and non-readily degradable molecules as found in: domestic wastewater, brewery wastewater, paper wastewater or the effluent of anaerobic digesters, have been used to generate electrical power in MFC. Unfortunately, power output from MFCs using such wastewaters is typically just 10% of the power generated from pure substrates. Moreover, the composition of wastewater has a large effect on the power output of MFCs.

Some researchers have found that spiking the wastewater with acetate results in an increase of the power output, indicating that the more readily biodegradable the substrate fraction within the wastewater, the higher the power output will be.

It is estimated that a wastewater treatment plant for 100,000 people has the potential to become a 2.3 MW power plant if all the energy is recovered as electricity. Therefore globally, the potential for electricity generation from municipal sewage and industrial wastewater is huge. However, the low concentration of the organic substrate in most wastewater presents a formidable challenge, since for efficient operation MFCs require sufficient substrate delivery to the anodic biofilm at rates sufficient to sustain the current generation. That is, a dilute substrate means that only a low current density is possible from MFCs, and large electrode area requirements are cost-prohibitive. The provision of a method for the preparation of a substrate from a wastewater source suitable for use in a MFC is therefore desirable.

It is therefore an aim of the present invention to provide a method for the recovery of organic compounds such as volatile fatty acids from wastewater in high concentration.

It is a further aim of the present invention to render the organic substrate readily biodegradable and suitable for use in a MFC.

According to a first aspect of the present invention there is provided a method of recovering organic compounds from wastewater for electricity generation in a microbial fuel cell, comprising the steps of:
i) converting organic compounds from wastewater into a biomass;
ii) recovering the biomass from the wastewater;
iii) followed by substantially dissolving or breaking down the biomass to form a cell lysate;
iv) fermenting the cell lysate to form volatile fatty acids (VFA) in a broth; and
v) separating the VFA in the VFA broth to produce a clarified VFA stream.

VFA are carboxylic acids with a carbon chain of six carbons or fewer.

The method of the present invention has the advantage that it is both simple to implement and may be integrated easily and readily into existing municipal sewage treatment works or industrial wastewater treatment plants. The method also overcomes the disadvantages of prior art methods described prior hereto.

It will be appreciated that the term ‘wastewater’ covers both sewage and industrial wastewater which are complex liquors and mainly comprise water with varying amounts of a wide-range of substances dispersed throughout their bulk. These substances vary greatly in terms of both chemical and physical properties. In addition, the substances also vary greatly in terms of their polluting effect if the substances enter a watercourse.

The ‘biological oxygen demand’ (BOD) of the wastewater represents the organic fraction useful for power generation.

The wastewater used in the present invention may be derived from: domestic wastewater and/or, municipal sewage and/or industrial wastewater.

Table 1 details a typical wastewater composition of sewage.

TABLE 1

| | |
|---|---|
| Biological Oxygen Demand, mg/L | 215 |
| Biological Oxygen Demand, mg/L | 430 |
| Suspended solids, mg/L | 251 |
| Ammonia, mg/L | 27 |
| Total phosphorus, mg/L | 14 |

The suspended solids in wastewater have a tendency to cause blockages in small flow channels of MFCs. Consequently, suspended solids may be preferably removed as a pre-treatment before the soluble organic substances are utilized, i.e. suspended solids present in the wastewater are preferably substantially removed before conversion of the organic compounds into biomass.

Suitable methods for suspended solids removal include one or more of: sedimentation tank, dissolved air flotation, filtration and centrifugation.

A preferred method of converting organic compounds from the wastewater into a biomass as in step (i) of the method of the invention is through biological treatment. In an aerobic biological treatment process, air is supplied to provide microorganisms that may then consume and convert the organic matter into biomass. Therefore in the method of the present invention the organic compounds may be preferably converted to biomass using an aerobic treatment process. Also in relation to the present invention the organic compounds from wastewater may be converted preferably into a biomass using an activated sludge process.

Nitrogen and phosphorus are also assimilated for biomass growth during the aerobic treatment. A suitable organic:nitrogen:phosphorus ratio for aerobic biological treatment is 100:5:1 based on theoretical calculations. In situations where the wastewater is deficient in either nitrogen or phosphorus or both, nutrient supplement is required for effective treatment. Therefore also in relation to the present invention it is preferred that the organic compounds are treated with nitrogen and phosphorus compounds.

The majority of municipal sewage treatment plants use activated sludge processes, wherein the process bacteria consume the biodegradable soluble organic contaminants (for example, sugars, fats, organic short-chain carbon molecules, and the like) and bind much of the less soluble fractions into ‘flocs’ as in the case of a suspended-growth system.

In a fixed-film system the ‘flocs’ are incorporated into a biofilm or slime layer that sloughs off when same becomes too thick. Flocs and broken biofilms are removed in a subsequent clarification step, which uses a sedimentation tank often called a secondary clarifier, secondary settling tank or humus tank.

The biological treatment regime has a significant influence on biomass yield.

Conventional wastewater treatment typically operates with a hydraulic retention time (HRT) of 5 to 10 hours and a biomass age (also known as sludge age) of 4 to 10 days, which provides a typical biomass yield of 0.5 kg biomass/kg BOD consumed as seen in Table 2.

Table 2 details typical biomass yield in different treatment regimes.

| Biological treatment | HRT | Biomass age | Biomass yield kg |
|---|---|---|---|
| regime | hours | days | biomass/kg BOD |
| High rate activated | 2.00 | 0.50 | 1.00 |
| sludge | | | |
| Conventional | 5.0-10.0 | 4.0-10.0 | 0.5 |
| operation | | | |
| Extended aeration | 20-48 | 24 | 0.25 |

For power generation, it is preferred to maximize the biomass yield and for this reason the HRT of the biological treatment process should not exceed 5 hours; preferably not more than 3 hours.

Therefore in relation to the method of present invention the organic compounds are preferably subject to a hydraulic retention time of 2 to 10 hours, and a biomass age of 0.5 to 10 days to achieve a biomass yield of 0.5 to 1 kg biomass per kg BOD consumed. More preferably, the organic compounds are subject to a maximum hydraulic retention time of 5 hours. Even more preferably, the organic compounds are subject to a maximum hydraulic retention time of 3 hours.

In step (ii) of the method of the invention the biomass is recovered. This recovery step may be effected using conventional means such as for example sedimentation tanks, dissolved air flotation, filtration and centrifugation.

In step (iii) of the method of the present invention the biomass is broken down or is dissolved to form a cell lysate. Bacterial biomass may be broken down by physical means such as grinding or sonication with an ultrasonic probe, or by treatment with caustic solution (NaOH), or by a combination of physical and chemical means.

In step (iv) of the method of the present invention the cell lysate is fermented to form volatile fatty acids (VFA) in a broth.

The process of converting biomass into VFA is well known as a natural degradation of organic matter under anaerobic conditions, a complex chain of biochemical reactions effected by several types of micro-organisms that require little or no oxygen.

The overall biochemical reactions may be summarized by Equation 2 as follows:

C₆H₁₃O₅+xH₂O→COOH—(CH₂)ₙ—CH₃→4CH₄+2CO₂ Equation 2.

By maintaining the bioreactor retention time to less than 6 days and maintaining a low pH, it is possible to suppress the methanogenic reactions and allow VFA to be recovered as the products of choice.

VFA may be produced from waste biomass including: municipal solid waste (MSW), municipal sewage sludge, and agricultural residues including manure. Products from industrial mixed-acid fermentation mainly comprise a mixture of acetic, propionic, butyric and pentanoic acids.

Hydrolysis is often the rate limiting step in VFA fermentation. Biomass is particularly difficult to break down. VFA yields in biomass conversion processes are usually limited to less than 20%. However, it has now been found that by breaking down or dissolving the biomass into a cell lysate before the fermentation, VFA yields of greater than 50%, for example greater than 80% may be achieved. Bacterial biomass may be broken down by physical means such as grinding or sonication with an ultrasonic probe, or by treatment with caustic solution (NaOH), or by a combination of physical and chemical means.

The fermented cell lysate or VFA broth contains low level of suspended solids. Nevertheless, any suspended solids have the potential to cause blockage of fine flow channels or settle on electrode surfaces and reduce electrical conductance. For this reason, it is desirable to minimize the presence of any suspended solids in the MFC feed. Thus any suspended solids present in the VFA broth are preferably removed. Suitable methods for suspended solids removal include: dissolved air flotation, centrifugation, filtration, dilution etc. Filtration by ultrafiltration membrane is particularly effective as it is capable of removing even colloidal species and macromolecules which may cause fouling of the anode.

Subsequently, the volatile fatty acids (VFA) are fed to a microbial fuel cell (MFC) or digester to generate electricity or biogas respectively.

Therefore, in accordance with a second aspect of the present invention there is provided the use of volatile fatty acids prepared using the method according to the first aspect of the present invention in a microbial fuel cell to generate electricity.

In accordance with a third aspect of the present invention there is provided a method of generating electricity comprising preparing volatile fatty acids (VFA) by the method according to the first aspect of the present invention and feeding the volatile fatty acids to a microbial fuel cell to generate electricity.

For the avoidance of doubt, all of the information detailed above in relation to the first aspect of the present invention also applies in relation to the second aspect of the present invention and also applied in relation to the third aspect of the present invention.

The microbial conversion of substrates is therefore a key process to generate electricity in MFCs. The type of substrate fed into a MFC has a significant impact on the structure and composition of the microbial community in the biofilm on the anode. The more reduced the substrate, the more energy is available for conversion to electricity and hence there is less need for a complex microbial community to establish in the MFC.

Without wishing to be bound by any particular theory, the inventor believes that the poor power outputs from MFC systems with complex wastewaters are due to large numbers of non-electron-producing bacteria in the biofilm which reduces the efficiency of the anode.

In addition, the accumulation of waste products in the biofilm, for example, oxidized intermediates or protons, needs to be prevented as this may change the redox conditions and hamper the metabolic activity of the biofilm.

A limited mass transfer of substrate or electron acceptors towards the anode or cathode respectively, may result in concentration or mass transfer losses. Substrate competing processes, such as fermentation or methanogenesis, result in a loss of electrons.

Also, part of the substrate may be inherently converted into anodophilic biomass. All these processes lower the conversion of substrate into current which is expressed by the coulombic efficiency (CE). The CE is defined as the ratio of the amount of substrate input to the amount of electrons recovered.

The present invention therefore overcomes many of the technical issues described in the foregoing by converting the biomass into volatile fatty acids (VFA), that is, as a readily biodegradable substrate, before the substrate is presented to the MFC. This means that many of the micro-organisms involved in the breakdown of complex molecules, their toxic waste products, and competing processes are prevented from interfering with the active biofilm on the anode.

For a better understanding of the present invention and to show more clearly how it may be carried into effect, the invention will now be described further by way of the following Figures and examples in which:

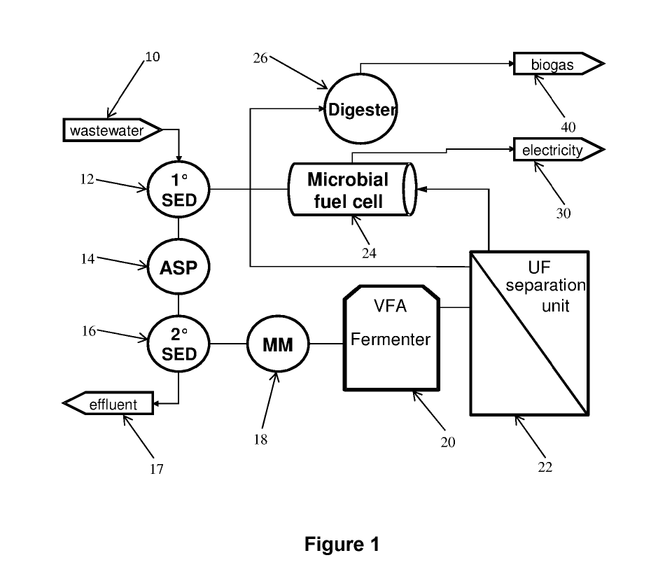

FIG. 1 illustrates a flow diagram of a system for the generation of electricity from wastewater using a microbial fuel cell in accordance with the present invention; and

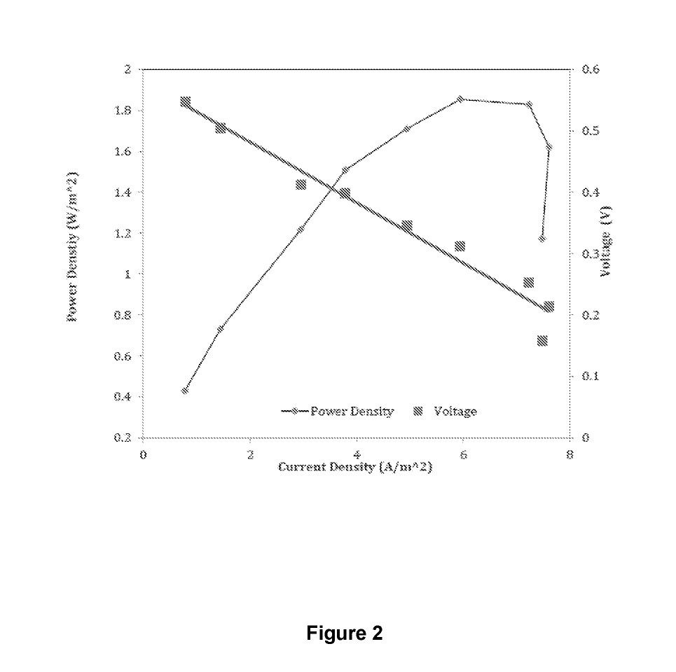

FIG. 2 is a graph illustrating the power output from a microbial fuel cell using volatile fatty acids as a substrate in accordance with the present invention.

### Experimental EXAMPLE 1

FIG. 1 illustrates an example of a conceptual system for the production of electricity from wastewater in accordance with the present invention for the preparation of volatile fatty acids (VFA) as a substrate for a microbial fuel cell (MFC), using organic compounds in wastewater.

In FIG. 1 wastewater (10) is fed to a first sedimentation tank (1° SED) (12) where any suspended solids are removed. Conversion of the organic compounds from wastewater into biomass takes place in the activated sludge process tank (ASP) (14), and separation of biomass from the wastewater takes place in the second sedimentation tank (2° SED) (16) with effluent (17) being removed. The cell lysate is generated by treating the biomass with a micro-mill grinder (MM) (18). The volatile fatty acid (VFA) substrate is generated by fermentation of the cell lysate in a VFA fermenter (20). An ultrafiltration (UF) (22) separation unit is used to produce a clarified VFA substrate solution which is then fed to a microbial fuel cell (24) for electricity production (30). A sludge stream from the first sedimentation tank and waste streams from both the UF separator and the MFC are combined to provide feed to a digester (26) where extra energy in the form of biogas (40) is generated.

### REFERENCE EXAMPLE 2

Samples of biomass from an activated sludge plant treating domestic sewage were taken for volatile fatty acid fermentation. The fermentation was performed in 5 L capacity glass bottles. The fermentation temperature was maintained at 35° C. using a water bath. The fermentation was conducted for a period of 96 hours. Fermented samples were analyzed daily to monitor the volatile fatty acid (VFA) generation during fermentation. Table 3 shows that the level of VFA in the fermentation increased rapidly during the first 24 hours but leveled out after 48 hours.

TABLE 3
Typical VFA yields in different treatment regimes.

| | | Dry | Soluble | | |
|---|---|---|---|---|---|
| Run Time | | Solids | COD | Ammonia | VFA |
| (hours) | pH | (% w/v) | (mg/L) | (mg/L) | (mg/L) |
| 0 | 7.02 | 1.67 | 450 | 50 | 10 |
| 24 | 6.35 | 1.55 | 5,310 | 480 | 1,984 |
| 48 | 6.16 | 1.40 | 5,700 | 600 | 2,695 |
| 72 | 6.33 | 1.36 | 5,930 | 610 | 2,775 |
| 96 | 6.45 | 1.31 | 6,240 | 660 | 2,920 |

### EXAMPLE 3

The experiment in example 2 was repeated except that all of the biomass samples were treated with 3% sodium hydroxide (NaOH) as a percentage of the total dry solids and milled in a micro-mill for 48 hours before fermentation. The average concentration of VFA in the resultant VFA broth was approximately 9,775 mg/L.

### REFERENCE EXAMPLE 4

The experiment in example 2 was again repeated, except that in example 4 all of the biomass samples were diluted with sludge from the first sedimentation tank in a 1:1 volume/volume ratio before fermentation. After fermentation, all samples were filtered through a Whatman GF/C filter (1.2 μm). The average results of the analyses of the filtered samples were as follows:

VFA concentration: approximately 3,800 mg/L;

Ammonia concentration: approximately 310 mg/L;

Phosphate concentration: approximately 40 mg/L;

Total soluble chemical oxygen demand (COD) concentration: approximately 8,000 mg/L.

The average VFA composition was as follows: Acetic (40%); Propionic (38%); Butyric (12%); Valeric (10%).

### EXAMPLE 5

A sample of the filtered VFA sample from Example 4 was used a substrate in a microbial fuel cell. FIG. 2 shows the power output from MFC trial which achieved an excellent power density based on surface area of 1850 mW/m².

Power density may be defined as the kWh generated per m³ of feed. Since the energy content of a unit volume of feed is proportional to the amount of VFA it contains, it follows that a stream with an increased level of VFA would produce a much higher power density than a stream with lower VFA levels.

Therefore, given the results of Example 5, it would be readily apparent to a skilled reader that using a clarified VFA stream prepared by the method of the present invention (and as illustrated in Example 3) in a microbial fuel cell would result in even greater power density values being obtained than for the stream obtained in examples 2 and 4′.

It will be appreciated that many modifications and enhancements may be made to the basic method outlined herein. For instance, the biomass may be substituted with waste biomass from other sources, for example waste biomass from pharmaceutical fermentation or a biorefinery. Other possible modifications will be readily apparent to the appropriately skilled person.

1. A method of recovering organic compounds from wastewater for use in electricity generation in a microbial fuel cell, comprising the steps of:
i) converting organic compounds from wastewater into a biomass;
ii) recovering the biomass from the wastewater;
iii) followed by substantially dissolving or breaking down the biomass to form a cell lysate;
iv) fermenting the cell lysate to form volatile fatty acids (VFA) in a broth; and
v) separating the VFA in the VFA broth to produce a clarified VFA stream.

2. A method according to claim 1 wherein the organic compounds from wastewater are converted to biomass using an aerobic treatment process.

3. A method according to claim 2 wherein the organic compounds from wastewater are converted to biomass using an activated sludge process.

4. A method according to claim 1, 2 or 3 wherein the wastewater is derived from: domestic wastewater and/or, municipal sewage and/or industrial wastewater.

5. A method according to any of the preceding claims wherein suspended solids present in the wastewater are substantially removed before conversion of the organic compounds into biomass.

6. A method according to claim 5 wherein the suspended solids are removed by using one or more of: a sedimentation tank, dissolved air flotation, filtration and centrifugation.

7. A method according to any of the preceding claims wherein the organic compounds are treated with nitrogen and phosphorus compounds.

8. A method according to any of the preceding claims wherein the organic compounds are subject to a hydraulic retention time of 2 to 10 hours, and a biomass age of 0.5 to 10 days to achieve a biomass yield of 0.5 to 1 kg biomass per kg BOD consumed.

9. A method according to claim 8 wherein the organic compounds are subject to a maximum hydraulic retention time of 5 hours.

10. A method according to claim 8 wherein the organic compounds are subject to a maximum hydraulic retention time of 3 hours.

11. A method according to any of claims 1 to 10 wherein the VFA yields are at least 50%.

12. A method according to any of claims 1 to 11 wherein suspended solids present in the VFA broth are removed.

13. A method according to any of claims 1 to 12 wherein the volatile fatty acids (VFA) are fed to a microbial fuel cell (MFC) or digester to generate electricity or biogas respectively.

14. Use of volatile fatty acids prepared using the method of claims 1 to 13 in a microbial fuel cell to generate electricity.

15. A method of generating electricity comprising preparing volatile fatty acids (VFA) by the method of claims 1 to 13 and feeding the volatile fatty acids to a microbial fuel cell to generate electricity.
